(19) United States
(12) Patent Application Publication
(10) Pub. No.: US 2008/0268542 A1
(43) Pub. Date: Oct. 30, 2008

(54) High Efficiency Electroporation Buffer

(75) Inventors: Teresa Rubio, El Cerrito, CA (US); Joseph Terefe, Rio Linda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/742,245

(22) Filed: Apr. 30, 2007

(51) Int. Cl. C07H 19/00; C12N 15/87

(52) U.S. Cl. 435/461; 536/22.1

(57) ABSTRACT

Electroporation of mammalian cells is performed to high efficiency by use of a buffer solution containing trehalose, sucrose, or both, in addition to an inorganic phosphate buffer, an organic sulfonic acid buffer, a halide salt of an alkali or alkaline earth metal, a chelating agent, an alkyl methyl sulfoxide, and a nucleotide triphosphate, with substantially no sodium. This buffer solution is effective both in the electroporation of cells that are suspended in the buffer solution and in the electroporation of cells that are fixed on a surface.

### BACKGROUND OF THE INVENTION

1. Field of the Invention

This invention lies in the field of transfection of membranous structures such as biological cells, liposomes, and vesicles with species that are exogenous to the structures. In particular, this invention addresses the buffer solutions through which the electric current used for the electroporation is transmitted, and how the choice of buffer solution affects the transfection efficiency and the ability of the membranous structures to survive the exposure to the applied voltage.

2. Description of the Prior Art

Transfection is of value to research biologists and biochemists in the performance of various investigations and procedures, including siRNA experiments, research using cDNA libraries, and other clinical and research studies. Electroporation is one of the most advanced transfection technologies and involves the application of electric field, typically in pulses, through a suspension of the membranous structures in a liquid solution of the exogenous species. It is believed that the electric field renders the membranes of the structures temporarily porous and thereby allows the species to penetrate the membrane.

As the value of transfection is increasingly recognized and it use expands, certain concerns have limited its applicability. One such concern is the efficiency of the procedure, i.e., the number of membranous structures that are successfully transfected with the exogenous species, and another is the ability of the membranous structures to remain intact and viable throughout the procedure or to spontaneously resume their intact condition at the completion of the procedure. One factor affecting both of these concerns is the composition of the buffer solution in which the structures are suspended, particularly in the case of structures that are biological cells. Various reports have shown the results of studies in which the electroporation buffer has been formulated to increase cell survival. One such report is that of van den Hoff, M. J. B., et al., “Electroporation in ‘intracellular’ buffer increases cell survival,” Nucleic Acids Research 20 (11), p. 2902 (1992), in which a buffer solution is used that is formulated to resemble the intracellular ionic composition. The buffer solution used by van den Hoff et al. contained 120 mM KCl, 0.15 mM CaCl₂, 10 mM K₂ HPO₄/KH₂PO₄, pH 7.6, 25 mM HEPES, pH 7.6, 2 mM EGTA, pH 7.6, 5 mM MgCl₂, and 2 mM ATP, pH 7.6, all pH's adjusted with KOH. Another approach is reported by Melkonyan, H., et al., “Electroporation efficiency in mammalian cells is increased by dimethyl sulfoxide (DMSO),” Nucleic Acids Research 24 (21), 4356-4357 (1996), in which dimethyl sulfoxide was used as an additive to an electroporation medium that otherwise consisted of RPMI supplemented with 10% FCS, where the DMSO constituted 1.25% by weight of the solution. A still further approach is that of Mussauer, H., et al., “Trehalose Improves Survival of Electrotransfected Mammalian Cells,” Cytometry 45: 161-169 (2001), in which the buffer was formulated by adding trehalose at varying concentrations to media that otherwise contained 0.85 mM K₂HPO₄, 0.3 mM KH₂PO₄ (pH 7.2), either 10 mM or 25 mM KCl, and inositol in amounts selected to achieve 100 or 150 mOsm (hypoosmolar conditions) or 290 mOsm (isoosmolar condition). Other reports are those of Hernádndez, J. L., et al., “A highly efficient electroporation method for the transfection of endothelial cells,” Angiogenesis 7: 235-241 (2004); Ovcharenko, D., et al., “High-throughput RNAi screening in vitro: From cell lines to primary cells,” RNA 11: 985-993 (2005); Golzio, G., et al., “Control by Osmotic Pressure of Voltage-Induced Permeabilization and Gene Transfer in Mammalian Cells,” Biophysical Journal 74: 3015-3022 (1998); \check{C}egovnik, U. et al., “Setting optimal parameters for in vitro electrotransfection of B16F1, SAI, LPB, SCK, L929 and CHO cells using predefined exponentially decaying electric pulses,” Bioelectrochemistry 62: 73-82 (2004); and Riemen, G., et al., United States Patent Application Publication No. US 2005/0064596 A1, filed Apr. 23, 2002 and published Mar. 24, 2005. The contents of each of the documents listed in this paragraph are incorporated herein by reference.

### SUMMARY OF THE INVENTION

A buffer solution has now been developed that is particularly effective in promoting a high degree of transfection during electroporation while in preserving cell integrity and viability. The buffer solution is of low ionic strength but contains a sugar component that allows the osmolarity of the buffer solution to be adjusted at various levels ranging from hyposmolar to isosmolar by adjusting the concentration of the sugar. The sugar used in the buffer solution is either trehalose, sucrose, or a combination of the two. For most cells, a hyposmolar condition is achieved by using a buffer with a sugar concentration of approximately 125 mM, while an isosmolar condition is achieved by using a buffer with a sugar concentration of approximately 275 mM. Other components are an inorganic phosphate buffer, an organic sulfonic acid buffer, a halide salt of an alkali or alkaline earth metal, a chelating agent, an alkyl methyl sulfoxide, and a nucleotide triphosphate. The buffer pH is preferably within the range of about 6.5 to about 7.7, and the buffer is substantially devoid of sodium ion. The term “substantially devoid” means that when sodium ion is present, the amount is so small that it does not cause a significant degree of cell damage, and is preferably in trace amounts at most.

The buffer solution of this invention is useful as a suspending medium for mammalian cells in an electroporation procedure performed on a suspension of the cells, and also as a solution through which electric pulses are transmitted in an electroporation procedure to mammalian cells that are grown on or otherwise attached to a surface.

### BRIEF DESCRIPTION OF THE FIGURES

FIG. 1 is a bar graph showing one set of results of electroporation experiments and comparing buffer solutions of the present invention with PBS.

FIG. 2 is a bar graph showing a second set of results of electroporation experiments and comparing buffer solutions of the present invention with PBS.

FIG. 3 is a bar graph showing a third set of results of electroporation experiments and comparing buffer solutions of the present invention with PBS.

### DETAILED DESCRIPTION OF THE INVENTION AND PREFERRED EMBODIMENTS

The amount of sugar in the buffer solution, whether the sugar be trehalose, sucrose, or a mixture of trehalose and sucrose, is one that will achieve the desired osmolarity, and as noted above, preferred total sugar concentrations are within the range of about 125 mM to about 300 mM, most preferably from about 150 mM to about 275 mM.

Examples of organic sulfonic acid buffers that can be used are 4-(2-hydroxyethyl)-1-piperazinylethanesulfonic acid (HEPES), 3-morpholinopropanesulfonic acid (MOPS), and 3-morpholinoethanesulfonic acid (MES). Among these, HEPES is the most preferred. The concentration of the organic sulfonic acid buffer can vary, but is preferably within the range of about 15 mM to about 50 mM, most preferably about 3 mM to about 10 mM and at a pH of about 7.2 to about 7.6.

The halide salt of an alkali or alkaline earth metal is a halide salt of a metal other than sodium, and a preferably a chloride salt. Magnesium chloride (MgCl₂) is particularly preferred. The concentration can vary, but is preferably one that will result in a total ionic strength that approximates the ionic strength of the mammalian cell. In preferred embodiments, the halide salt concentration is from about 1 mM to about 20 mM, most preferably from about 1 mM to about 10 mM.

The chelating agent is one that forms a chelate complex with the alkali or alkaline earth metal that is the cation of the halide salt. Examples of suitable chelating agents are ethylenediamine tetraacetic acid (EDTA) and ethylene glycol tetraacetic acid (EGTA). Among these, EGTA is preferred. The concentration of the chelating agent can vary as well, but will generally be in a chelating amount for the cation, i.e., an amount sufficient to produce chelate complexes of substantially the entire amount of metal cation present. In preferred embodiments, the amount is within the range of about 1 mM to about 20 mM, and most preferably from about 1 mM to about 5 mM.

The alkyl methyl sulfoxide is preferably a straight-chain C₁-C₄ alkyl methyl sulfoxide, a prime example of which is dimethyl sulfoxide (DMSO). The alkyl methyl sulfoxide is preferably included in an amount that will enhance the cell permeability of the exogenous species, and within this goal its concentration can vary. Preferred concentrations are within the range of about 0.3% to about 3% by weight.

The nucleotide triphosphate is included in an amount that will help preserve the viability of the cell, and in most cases will be within the range of about 1 mM to about 10 mM. A preferred nucleotide triphosphate is adenosine triphosphate.

The pH of the buffer solution can range from 6.5 to 7.7, maintained by the phosphate buffer. The buffer solution may also contain further components, an example of which is glutathione. When glutathione is present, its concentration preferably ranges from about 1 mM to about 10 mM, most preferably from about 1 mM to about 3 mM. The optimal amount will be an amount that will enhance the preservation of the cells, and the need for this component and its amount will vary with the choice of cells that are being transfected. Preferred buffer solutions are those that contain only the components listed above and no additional components.

Once all components have been incorporated into the buffer solution, the pH of the solution can be adjusted with KOH. The conductivity of the solution can range from about 2 mS/cm to about 4.2 mS/cm. Transfection is then performed by conventional procedures known in the art, using conventional equipment and instrumentation. The following examples are offered strictly for purposes of illustration.

### EXAMPLES

Stock solutions of the individual components for test electroporation buffers were prepared as follows. A 0.2 M potassium phosphate buffer was prepared by dissolving 86.6 mL of K₂HPO₄ (1M, Catalog No. 60354-1Kg) and 13.4 mL of KH₂PO₄ (1M, Catalog No. P5655-1Kg) in 500 mL of water. A 1M solution of sucrose was prepared by dissolving 171.15 g of sucrose in 500 mL of water. A 1M solution of trehalose was prepared by dissolving 94.57 g of trehalose in 250 mL of water. HEPES was obtained from Sigma, Catalog No. H0887 at 1M. Magnesium chloride was also obtained from Sigma, Catalog No. M1028 at 1M. EGTA was likewise obtained from Sigma, Catalog No. E4318-10G, and a 0.5M solution was prepared by dissolving 4.7 g in 25 mL of water, adding 10 mL of water with 10N KOH to a pH of 8. The solution was stored at −20° C. Undiluted DMSO was likewise obtained from Sigma, Catalog No. 02650-100H.

A series of test buffers were prepared with the compositions shown in Table I.

TABLE I
Test Buffer Compositions
All concentrations are in mM except DMSO which is expressed in weight percent.

| | No. | | | | | | | | |
|---|---|---|---|---|---|---|---|---|---|
| Component | (1) | (2) | (3) | (4) | (5) | (6) | (7) | (8) | (9) |
| Trehalose | — | — | — | — | 250 | 150 | — | — | — |
| Sucrose | 250 | 250 | 150 | 150 | — | — | 150 | 150 | 150 |
| Potassium | 5 | 5 | 5 | 5 | 5 | 5 | 5 | 5 | 5 |
| phosphate | | | | | | | | | |
| HEPES | 25 | 25 | 25 | 25 | 25 | 25 | 25 | 25 | 25 |
| MgCl₂ | 5 | 5 | 5 | 5 | 5 | 5 | 5 | 5 | 5 |
| EGTA | 2 | 2 | 2 | 2 | 2 | 2 | 2 | 2 | 2 |
| DMSO | 1% | 1% | 1% | 1% | 1% | 1% | 1% | 1% | — |
| ATP | 2 | 2 | — | 2 | 2 | 2 | 2 | 2 | — |
| Glutathione | — | — | 2 | 2 | — | — | — | — | — |
| pH | 6.9-7.1 | 7.2-7.5 | 7.2-7.5 | 7.2-7.5 | 7.2-7.5 | 7.2-7.5 | 7.2-7.5 | 7.2-7.5 | 7.2-7.5 |

Electroporation was performed using each of these buffer solutions, and comparing them to phosphate-buffered saline (PBS, Invitrogen Catalog No. 14190-144). The cells were Cos7 cells (SV40 transformed kidney cells) and 5F2C cells (a CHO cell line stably transformed with the luciferase gene), and the species with which the cells were transfected was either plasmid DNA (pCMVi-Luc) or siRNA. A general procedure and conditions were as follows:

Harvesting and counting the cells. The cells are passaged the day before electroporation to place them in actively growing condition for harvesting. To achieve this condition with adherent cells, the cells are trypsinized to detach the cells from the surface, growth media is added, and the cells are pelleted. To achieve this condition with suspended cells, the cells are simply pelleted. In both cases, the media is removed after pelleting, and the cells are then washed once with PBS by carefully pipetting the cells. An aliquot is taken and then counted.

Preparing the cells for electroporation. An aliquot is drawn that contains the number of cells needed to perform the experiment. For adherent cells, a recommended aliquot contains 1×10⁶ cells/mL, but can be within the range of 0.01-20×10⁶ cells/mL. For suspension cells, a recommended aliquot is one containing 2-3×10⁶ cells/mL. In either case, the cells in the aliquot are then pelleted, the PBS is aspirated, and the pelleted cells are resuspended in the appropriate volume of electroporation buffer reagent (1 mL per 1×10⁶ of adherent cells, and 1 mL per 2-3×10⁶ of suspension cells). The exogenous species, which in these experiments was a nucleic acid, is then added at an appropriate concentration. For siRNA, 5-500 nM can be used. For plasmid DNA, 1-100 μg/mL can be used.

Electroporation. A cuvette that is 0.2 cm in size is charged with 100-200 μL of the suspension containing the cells in the electroporation buffer reagent with the nucleic acid. For a 0.4 cm cuvette, 400-800 μL of the suspension is used. Electroporation is then performed at optimal conditions. The cells are then transferred to tissue culture dishes containing growth media where they are incubated at 37° C. in a humidified CO₂ incubator until assayed. The growth media is changed every 24 hours.

Assessing Transfection Efficiency. Fluorescently labeled siRNAs can be used to determine the transfection efficiency for siRNA delivery. Transfection efficiency can be measured by fluorescent microscopy or by flow-cytometry. For plasmid delivery, transfection efficiency can be determined by use of plasmids expressing reporter genes such as GFP (green fluorescent protein), luciferase, or β-galactosidase.

The Cos7 cells were transfected with the plasmid pCMVi-Luc that expresses the luciferase gene. Twenty-four hours after transfection, the cells were lysed by incubation for 15-20 minutes at 40° C. in 0.1 M phosphate buffer at pH 7.8 containing 1% Triton X-100, 2 mM EDTA, and 1 mM DTT. A portion of the lysate was then loaded onto a luminometer plate and the plate was placed in a luminometer that automatically added 100 μL of Reagent A which consisted of 3 mM ATP, 15 mM MgSO₄, 30 mM Tricine buffer, and 10 mM DTT, pH 7.8. Reagent B, 100 μL, consisting of 1 mM LH2 (luciferine) and deionized water, pH 6.0-6.4, was then added, and the light output was measured for 10-30 seconds.

The 5F2C cells were transfected in two groups, each with one of two siRNAs—a scramble negative control and a luciferase siRNA that silencea the luciferase gene. The degree of silencing, expressed in reference to the scramble transfections, was measured in the same manner as in the Cos7 cell experiments above, and was used as a measure of the transfection efficiency.

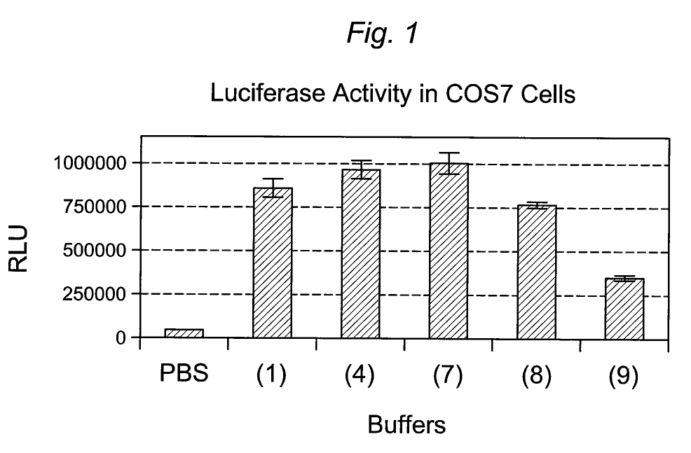

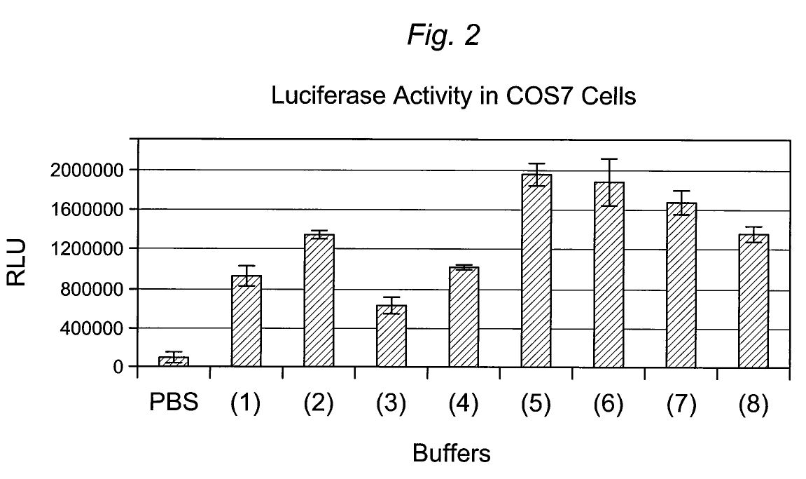

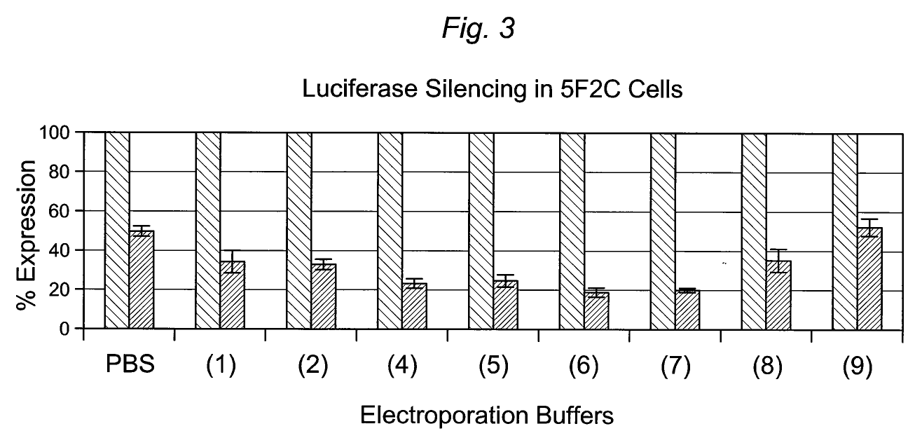

The results for all test buffers listed in Table I, in various combinations, are shown in the Figures. Relative light fluorescence units (RLU) for luciferase activity in the transfected Cos7 cells for one set of experiments is shown in FIG. 1 and for another set in FIG. 2; and the luciferase silencing in the transfected 5F2C cells is shown in FIG. 3, where two bars are shown for each test buffer solution, the left bar representing transfection with the scramble negative control and the right bar representing transfection with the luciferase siRNA. The results show that all, or essentially all, of the test buffers were superior in performance to PBS. In terms of pH, the best performance was achieved with a pH above 7.2, and specifically in the range of 7.2-7.5-compare buffer (2) with buffer (1). In terms of the presence of ATP and glutathione, the best performance was achieved with ATP alone—compare buffers (5), (6), and (7) with buffers (3) and (4). Also, buffers containing all six components, i.e., the sugar, potassium phosphate, HEPES, MgCl₂, EGTA, DMSO, and ATP, were superior to buffers lacking one of the components (DMSO)—compare buffers (5), (6), and (7) with buffers (8) and (9).

In the claims appended hereto, the term “a” or “an” is intended to mean “one or more,” and the term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A buffer solution for use as an electric pulse transmitting agent for mammalian cells and as a solvent for species exogenous to said cells in an electroporation procedure in which said cells are transfected with said exogenous species, said buffer solution comprising:
(i) a sugar selected from the group consisting of trehalose and sucrose in a concentration that is at least approximately isotonic relative to said cells;
(ii) an inorganic phosphate buffer at a pH of from about 6.5 to about 7.7;
(iii) an organic sulfonic acid buffer in a buffering amount;
(iv) a halide salt of an alkali or alkaline earth metal other than sodium in an amount that promotes transfection;
(v) a chelating agent in a chelating amount for said alkali or alkaline earth metal;
(vi) an alkyl methyl sulfoxide in a cell permeability enhancing amount; and
(vii) a nucleotide triphosphate in a cell preserving amount;
said buffer solution being substantially devoid of sodium ion.

2. The buffer solution of claim 1 wherein said sugar concentration is from about 125 mM to about 300 mM.

3. The buffer solution of claim 1 wherein said organic sulfonic acid buffer is a member selected from the group consisting of 4-(2-hydroxyethyl)-1-piperazinylethanesulfonic acid, 3-morpholinopropanesulfonic acid, and 3-morpholinoethanesulfonic acid, and is at a concentration of from about 15 mM to about 50 mM.

4. The buffer solution of claim 1 wherein said halide salt of an alkali or alkaline earth metal other than sodium is a chloride salt and is at a concentration of from about 1 mM to about 20 mM.

5. The buffer solution of claim 1 wherein said halide salt of an alkali or alkaline earth metal other than sodium is magnesium chloride and is at a concentration of from about 1 mM to about 10 mM.

6. The buffer solution of claim 1 wherein said chelating agent is a member selected from the group consisting of ethylenediamine tetraacetic acid and ethylene glycol tetraacetic acid, and is at a concentration of from about 1 mM to about 20 mM.

7. The buffer solution of claim 1 wherein said chelating agent is ethylene glycol tetraacetic acid and is at a concentration of from about 1 mM to about 5 mM.

8. The buffer solution of claim 1 wherein said alkyl methyl sulfoxide is dimethyl sulfoxide and is present at a concentration of from about 0.3% to about 3%, by weight.

9. The buffer solution of claim 1 wherein said nucleotide triphosphate is adenosine triphosphate at a concentration of from about 1 mM to about 10 mM.

10. The buffer solution of claim 1 wherein said sugar is trehalose at about 125 mM to about 300 mM, said inorganic phosphate buffer is potassium phosphate at a pH of from about 7.2 to about 7.6 and a concentration of about 3 mM to about 10 mM, said organic sulfonic acid buffer is 4-(2-hydroxyethyl)-1-piperazinylethanesulfonic acid at about 15 mM to about 50 mM, said halide salt of an alkali or alkaline earth metal other than sodium is magnesium chloride at about 1 mM to about 10 mM, said chelating agent is ethylene glycol tetraacetic acid at about 1 mM to about 5 mM, and said alkyl methyl sulfoxide is dimethyl sulfoxide and is at a concentration of from about 0.3% to about 3% by weight.

11. The buffer solution of claim 1 wherein said sugar is sucrose at about 125 mM to about 300 mM, said inorganic phosphate buffer is potassium phosphate at a pH of from about 7.2 to about 7.6 and a concentration of about 3 mM to about 10 mM, said organic sulfonic acid buffer is 4-(2-hydroxyethyl)-1-piperazinylethanesulfonic acid at about 15 mM to about 50 mM, said halide salt of an alkali or alkaline earth metal other than sodium is magnesium chloride at about 1 mM to about 10 mM, said chelating agent is ethylene glycol tetraacetic acid at about 1 mM to about 5 mM, and said alkyl methyl sulfoxide is dimethyl sulfoxide and is at a concentration of from about 0.3% to about 3% by weight.

12. A process for transfecting mammalian cells with exogenous species by electroporation, said process comprising:
(a) contacting said cells with a solution of said exogenous species in a buffer solution comprising:
(i) a sugar selected from the group consisting of trehalose and sucrose in a concentration that is at least approximately isotonic relative to said cells;
(ii) an inorganic phosphate buffer at a pH of from about 7.2 to about 7.6;
(iii) an organic sulfonic acid buffer in a buffering amount;
(iv) a halide salt of an alkali or alkaline earth metal other than sodium in an amount that promotes transfection;
(v) a chelating agent in a chelating amount for said alkali or alkaline earth metal;
(vi) an alkyl methyl sulfoxide in a cell permeability enhancing amount; and
(vii) a nucleotide triphosphate in a cell preserving amount;
said buffer solution being substantially devoid of sodium ion; and
(b) applying a pulsewise electric voltage to said solution to achieve transfection of said cells with said exogenous species.

13. The process of claim 12 wherein said sugar concentration is from about 125 mM to about 300 mM.

14. The process of claim 12 wherein said organic sulfonic acid buffer is a member selected from the group consisting of 4-(2-hydroxyethyl)-1-piperazinyl-ethanesulfonic acid, 3-morpholinopropanesulfonic acid, and 3-morpholinoethanesulfonic acid, and is at a concentration of from about 15 mM to about 50 mM.

15. The process of claim 12 wherein said halide salt of an alkali or alkaline earth metal other than sodium is a chloride salt and is at a concentration of from about 1 mM to about 20 mM.

16. The process of claim 12 wherein said halide salt of an alkali or alkaline earth metal other than sodium is magnesium chloride and is at a concentration of from about 1 mM to about 10 mM.

17. The process of claim 12 wherein said chelating agent is a member selected from the group consisting of ethylenediamine tetraacetic acid and ethylene glycol tetraacetic acid, and is at a concentration of from about 1 mM to about 20 mM.

18. The process of claim 12 wherein said chelating agent is ethylene glycol tetraacetic acid and is at a concentration of from about 1 mM to about 5 mM.

19. The process of claim 12 wherein said alkyl methyl sulfoxide is dimethyl sulfoxide and is present at a concentration of from about 0.3% to about 3%, by weight.

20. The process of claim 12 wherein said nucleotide triphosphate is adenosine triphosphate at a concentration of from about 1 mM to about 10 mM.

21. The process of claim 12 wherein said sugar is trehalose at about 125 mM to about 300 mM, said inorganic phosphate buffer is potassium phosphate at a pH of from about 7.2 to about 7.6 and a concentration of about 3 mM to about 10 mM, said organic sulfonic acid buffer is 4-(2-hydroxyethyl)-1-piperazinylethanesulfonic acid at about 15 mM to about 50 mM, said halide salt of an alkali or alkaline earth metal other than sodium is magnesium chloride at about 1 mM to about 10 mM, said chelating agent is ethylene glycol tetraacetic acid at about 1 mM to about 5 mM, and said alkyl methyl sulfoxide is dimethyl sulfoxide and is at a concentration of from about 0.3% to about 3% by weight.

22. The process of claim 12 wherein said sugar is sucrose at about 125 mM to about 300 mM, said inorganic phosphate buffer is potassium phosphate at a pH of from about 7.2 to about 7.6 and a concentration of about 3 mM to about 10 mM, said organic sulfonic acid buffer is 4-(2-hydroxyethyl)-1-piperazinylethanesulfonic acid at about 15 mM to about 50 mM, said halide salt of an alkali or alkaline earth metal other than sodium is magnesium chloride at about 1 mM to about 10 mM, said chelating agent is ethylene glycol tetraacetic acid at about 1 mM to about 5 mM, and said alkyl methyl sulfoxide is dimethyl sulfoxide and is at a concentration of from about 0.3% to about 3% by weight.
